(19) United States
(12) Patent Application Publication
(10) Pub. No.: US 2021/0170367 A1
(43) Pub. Date: Jun. 10, 2021

(54) METATHESIS CATALYSTS AND METHODS OF PRODUCING PROPENE

(71) Applicant: Saudi Arabian Oil Company, Dhahran (SA)

(72) Inventors: Maha Alsayegh, Khobar (SA); Munir D. Khokhar, Dhahran (SA)

(73) Assignee: Saudi Arabian Oil Company, Dhahran (SA)

(21) Appl. No.: 16/704,154

(22) Filed: Dec. 5, 2019

(51) Int. Cl. B01J 23/30; B01J 21/18; B01J 35/10; B01J 35/02; B01J 37/02; C07C 6/04

(52) U.S. Cl. CPC B01J 23/30; B01J 21/18; B01J 35/1023; B01J 35/023; C07C 2521/18; B01J 35/1042; B01J 37/0201; C07C 6/04; C07C 2523/30; B01J 35/1038

(57) ABSTRACT

The present disclosure includes a metathesis catalyst comprising 80 weight percent to 99 weight percent catalyst support material and 1 weight percent to 20 weight percent catalytically active compound, based on the total weight of the metathesis catalyst. The catalyst support material may comprise carbon. The catalytically active compound may comprise tungsten and be supported by the catalyst support material. Methods of producing olefins through the metathesis of butene using the metathesis catalyst is also disclosed.

### TECHNICAL FIELD

The present disclosure generally relates to catalyst compositions and, more specifically, to metathesis catalysts and methods of producing olefins through the metathesis of butene using the metathesis catalysts.

### BACKGROUND

In recent years, there has been a dramatic increase in the demand for propene to supply the growing markets for polypropylene, propylene oxide, and acrylic acid. Currently, most of the propene produced worldwide (approximately 74 million tons/year) is a by-product from steam cracking units (57%), which primarily produce ethylene, or a by-product from Fluid Catalytic Cracking (FCC) units (30%), which primarily produce gasoline. However, these processes cannot respond adequately to the rapid increase in propene demand. As a result, alternative methods to directly produce propene have been developed and, in particular, methods of directly producing propene from feedstocks comprising butene, such as raffinate streams, have been developed.

The production of propene from feedstocks comprising butene can be accomplished through the metathesis of the butene to propene and other olefin compounds. The metathesis of butene to produce propene can better meet the growing demand for propene. However, due to the inefficiencies of conventional metathesis catalysts and reactor systems, the production of propene continues to present many difficulties. For example, in order to promote particular metathesis reactions, such as the self-metathesis of butene, it may be necessary to heat catalysts to relatively high temperatures, such as temperatures greater than 400 degrees Celsius (° C.). This may require the use of large furnaces and, as a result, a number of limitations may be encountered power during the operation of the reactor system. These limitations may include relatively slow heating rates due to the time required to transfer heat from the furnace to the catalyst and, as a result, increased energy consumption by the system.

### SUMMARY

Accordingly, there is an ongoing need for metathesis catalysts and methods of producing propene, which increase the efficiency of the metathesis process. The present disclosure is directed to metathesis catalysts that comprise an electrically conductive catalyst support material. These catalyst support materials may allow for the efficient heating of the metathesis catalyst via joule heating. This may greatly reduce the energy consumed to heat the metathesis catalysts and, as a result, increase the overall efficiency of methods of producing propene that utilize such metathesis catalysts.

According to one or more embodiments of the present disclosure, a metathesis catalyst may comprise 80 weight percent to 99 weight percent catalyst support material and 1 weight percent to 20 weight percent catalytically active compound, based on the total weight of the metathesis catalyst. The catalyst support material may comprise carbon. The catalytically active compound may comprise tungsten and may be supported by the catalyst support material.

According to one or more additional embodiments of the present disclosure, a method of producing propene from a feedstock comprising butene may comprise heating a metathesis catalyst to an operational temperature and contacting the feedstock with the metathesis catalyst. The metathesis catalyst may comprise 80 weight percent to 99 weight percent catalyst support material and 1 weight percent to 20 weight percent catalytically active compound, based on the total weight of the metathesis catalyst. The catalyst support material may comprise carbon. The catalytically active compound may comprise tungsten and may be supported by the catalyst support material. The contacting of the feedstock with the metathesis catalyst at the operational temperature may result in at least a portion of the butene in the feedstock to undergo a metathesis reaction and produce propene.

According to yet another embodiment of the present disclosure, a method of producing propene from a feedstock comprising butene may comprise joule heating a metathesis catalyst to an operational temperature and contacting the feedstock with the metathesis catalyst. The contacting of the feedstock with the metathesis catalyst at the operational temperature may result in at least a portion of the butene in the feedstock to undergo a metathesis reaction and produce propene.

Additional features and advantages of the embodiments of the present disclosure will be set forth in the detailed description that follows and, in part, will be readily apparent to those skilled in the art from the detailed description or recognized by practicing the embodiments of the present disclosure, as described in the detailed description.

### BRIEF DESCRIPTION OF THE DRAWINGS

The following detailed description of the embodiments of the present disclosure can be best understood when read in conjunction with the following drawings, where like structure is indicated with like reference numerals and in which:

FIG. 1 schematically depicts a fixed-bed continuous-flow reactor including a metathesis reaction zone, according to one or more embodiments of the present disclosure;

FIG. 2 schematically depicts another fixed-bed continuous-flow reactor including a metathesis reaction zone, according to one or more embodiments of the present disclosure;

FIG. 3 schematically depicts a reaction apparatus capable of joule heating a metathesis catalyst sample, according to one or more embodiments of the present disclosure;

FIG. 4 graphically depicts the temperature of a metathesis catalyst (y-axis) as a function of power applied to the metathesis catalyst (x-axis), according to one or more embodiments of the present disclosure;

FIG. 5A graphically depicts the X-ray diffraction (XRD) profile of silica, according to one or more embodiments of the present disclosure;

FIG. 5B graphically depicts the XRD profile of activated carbon, according to one or more embodiments of the present disclosure;

FIG. 6 graphically depicts the XRD profiles of tungsten oxide and tungsten oxide deposited on a silica support material, according to one or more embodiments of the present disclosure; and

FIG. 7 graphically depicts the XRD profiles of tungsten oxide deposited on an activated carbon powder support material and tungsten oxide deposited on an activated carbon bead support material, according to one or more embodiments of the present disclosure.

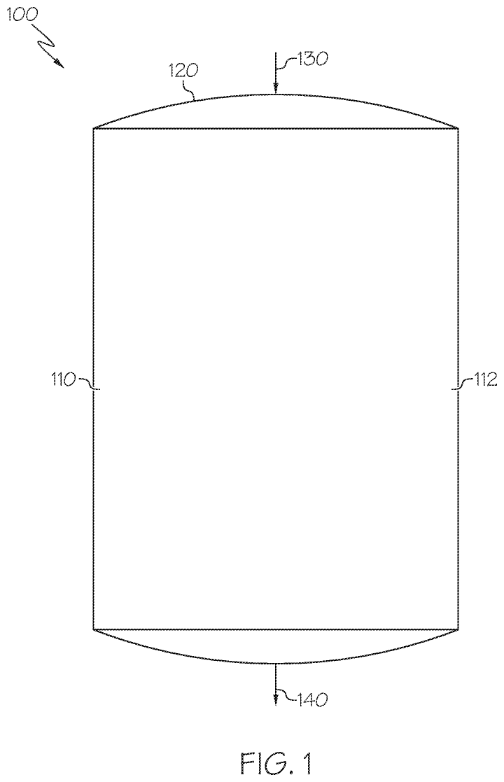

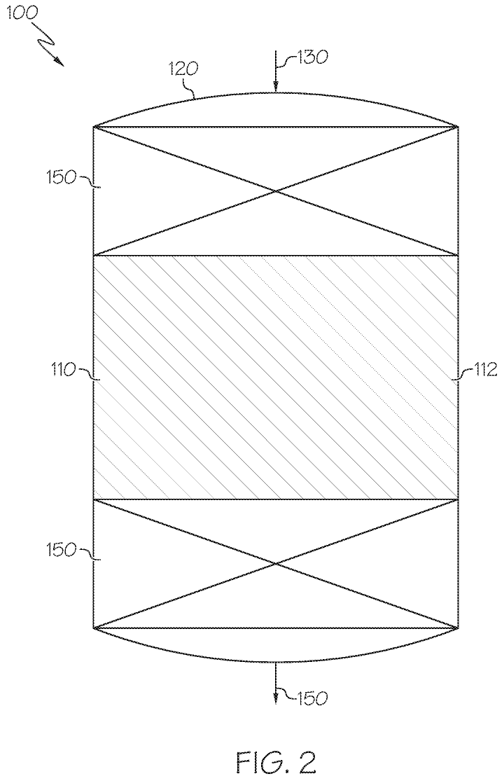

For the purpose of describing the simplified schematic illustrations and descriptions of FIGS. 1 and 2, the numerous valves, temperature sensors, electronic controllers, and the like that may be employed and well-known to those of ordinary skill in the art are not included. Further, accompanying components that are often included in typical chemical processing operations, such as gas supply systems, pumps, compressors, furnaces, or other subsystems, are not depicted. However, it should be understood that these components are within the spirit and scope of the embodiments of the present disclosure.

Additionally, for the purpose of describing the simplified schematic illustrations and descriptions of FIGS. 1 and 2, various process streams may be depicted as arrows. However, the arrows may equivalently refer to transfer lines, which may serve to transfer process streams between two or more system components. Arrows that connect to system components may also define inlets or outlets in each given system component. Arrows that do not connect two or more system components may signify a product stream that exits the depicted system or a system inlet stream that enters the depicted system. The arrow direction generally corresponds with the major direction of movement of the process stream or the process stream contained within the physical transfer line signified by the arrow.

Further, for the purpose of describing the simplified schematic illustrations and descriptions of FIGS. 1 and 2, arrows may schematically depict process steps of transporting a process stream from one system component to another system component. For example, an arrow from one system component pointing to another system component may represent “passing” a system component effluent to another system component, which may include the contents of a process stream “exiting” or being “removed” from one system component and “introducing” the contents of the process stream to another system component.

Reference will now be made in greater detail to various embodiments, some of which are illustrated in the accompanying drawings.

### DETAILED DESCRIPTION

As noted previously, the present disclosure is directed to metathesis catalysts and methods of producing propene by the metathesis of butenes. In particular, the present disclosure is directed to a metathesis catalyst comprising a catalyst support material and a catalytically active compound supported by the catalyst support material. The present disclosure is also directed to methods of producing propene from a feedstock comprising butene. The methods may comprise heating the metathesis catalysts of the present disclosure to an operational temperature and contacting the feedstock with the metathesis catalysts. The methods may also comprise joule heating a metathesis catalyst to an operational temperature and contacting the feedstock with the metathesis catalysts.

As used throughout the present disclosure, the term “butene” or “butenes” may refer to compositions comprising 1-butene, 2-butene, isobutene, or combinations of two or more of these isomers. Similarly, the term “2-butene” may refer to compositions comprising trans-2-butene, cis-2-butene, or combinations of these isomers.

As used in the present disclosure, the term “catalyst” may refer to a solid particulate comprising at least a catalyst support material and at least one catalytically active compound. The term “catalytically active compound” may refer to any substance that increases the rate of a particular chemical reaction. Catalytically active compounds and catalysts comprising the catalytically active compounds described in the present disclosure may be utilized to promote various reactions, such as, but not limited to, isomerization, metathesis, cracking, or combinations of these. The term “catalytic activity” may refer to a degree to which the catalyst increases the reaction rate of a reaction. For example, greater catalytic activity of a catalyst may increase the reaction rate of a reaction compared to a catalyst having a lesser catalytic activity.

As shown in Reaction 1 (RXN 1), the cross-metathesis of 1-butene and 2-butene may produce 1-propene and 2-pentene. As used in the present disclosure, the term “cross-metathesis” may refer to an organic reaction that involves the redistribution of fragments of alkenes by the scission and regeneration of carbon-carbon double bonds. In the case of metathesis between 2-butene and 1-butene, the redistribution of these carbon-carbon double bonds through metathesis produces propene and C5-C₆ olefins. The cross-metathesis of 1-butene and 2-butene may be achieved with a metathesis catalyst. As used in the present disclosure, the term “metathesis catalyst” may refer to a catalyst that promotes the metathesis reaction of alkenes to form other alkenes. The metathesis catalyst may also isomerize 2-butenes to 1-butene through a “self-metathesis” reaction mechanism, as shown in Reaction 2 (RXN 2).

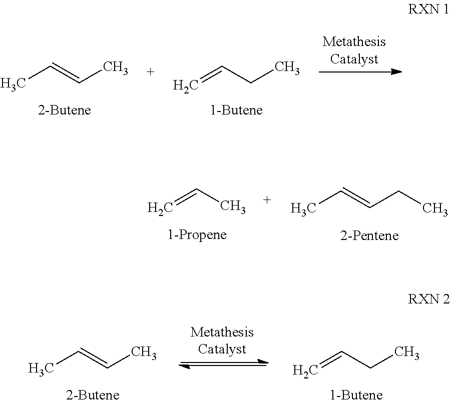

Referring to RXN 1 and RXN 2, the metathesis reactions are not limited to these reactants and products; however, RXN 1 and RXN 2 provide a simplified illustration of the reaction methodology. As shown in RXN 1, metathesis reactions may take place between two alkenes. The groups bonded to the carbon atoms of the carbon-carbon double bond may be exchanged between the molecules to produce two new alkenes with the exchanged groups. The specific metathesis catalyst that is selected may generally determine whether a cis-isomer or trans-isomer is formed, as the formation of a cis-isomer or a trans-isomer may be, at least partially, a function of the coordination of the alkenes with the catalyst.

As shown in RXN 1 and RXN 2, the metathesis of butenes may be promoted by a metathesis catalyst. The metathesis catalysts may generally comprise one or more components operable to promote the self-metathesis of butene, the cross-metathesis of butene, or combinations of these. For example, the metathesis catalysts may comprise an electrically conductive catalyst support material and a catalytically active compound, such as a catalytically active compound supported by an electrically conductive catalyst support material. The catalyst support may include activated carbon in various forms that facilitate the conduction of an electric current, such as activated carbon beads. As used throughout the present disclosure, the term “activated carbon” may refer to carbon that has been processed to comprise small, low-volume pores and, as a result, an increased surface area. Without being bound by any particular theory, it is believed that activated carbon may allow the metathesis catalyst to be heated via joule heating. That is, due to the electric and thermal conductivity of the activated carbon, a metathesis catalyst comprising such a catalyst support material may be heated by passing an electric current through the catalyst support material. As mentioned previously and described in further detail subsequently, the heating of the metathesis catalyst via joule heating may significantly increase the efficiency of metathesis processes.

The catalyst support material may have an average particle size of from 1 millimeter (mm) to 5 mm, as determined by scanning electron microscopy (SEM). For example, the catalyst support material may have an average particle size of from 1 mm to 4.5 mm, from 1 mm to 4 mm, from 1 mm to 3.5 mm, from 1 mm to 3 mm, from 1 mm to 2.5 mm, from 1 mm to 2 mm, from 1 mm to 1.5 mm, from 1.5 mm to 5 mm, from 1.5 mm to 4.5 mm, from 1.5 mm to 4 mm, from 1.5 mm to 3.5 mm, from 1.5 mm to 3 mm, from 1.5 mm to 2.5 mm, from 1.5 mm to 2 mm, from 2 mm to 5 mm, from 2 mm to 4.5 mm, from 2 mm to 4 mm, from 2 mm to 3.5 mm, from 2 mm to 3 mm, from 2 mm to 2.5 mm, from 2.5 mm to 5 mm, from 2.5 mm to 4.5 mm, from 2.5 mm to 4 mm, from 2.5 mm to 3.5 mm, from 2.5 mm to 3 mm, from 3 mm to 5 mm, from 3 mm to 4.5 mm, from 3 mm to 4 mm, from 3 mm to 3.5 mm, from 3.5 mm to 5 mm, from 3.5 mm to 4.5 mm, from 3.5 mm to 4 mm, from 4 mm to 5 mm, from 4 mm to 4.5 mm, or from 4.5 mm to 5 mm, as determined by SEM. Catalyst support materials having an average particle size less than 1 mm may pack too tightly and, as a result, may restrict the flow of gas and feedstock through the reactor and cause a pressure drop during operation of the reactor. Moreover, catalyst support materials having average particle sizes greater than 5 mm may not have sufficient contact between particles and, as a result, fail to conduct an electric current. The inability to conduct an electric current may reduce or prevent the heating of the metathesis catalysts via joule heating, as described subsequently in the present disclosure.

The catalyst support material may have an electrical conductivity sufficient to allow the catalyst support material to be heated via joule heating, as described subsequently in the present disclosure. Accordingly, the catalyst support material may have an electrical conductivity of from 80 Siemens per meter (S/m) to 7500 S/m at 20° C. For example, the catalyst support material may have an electrical conductivity of from 50 S/m to 6500 S/m, from 50 S/m to 5500 S/m, from 50 S/m to 4500 S/m, from 50 S/m to 3500 S/m, from 50 S/m to 2500 S/m, from 50 S/m to 1500 S/m, from 50 S/m to 500 S/m, from 500 S/m to 7500 S/m, from 500 S/m to 6500 S/m, from 500 S/m to 5500 S/m, from 500 S/m to 4500 S/m, from 500 S/m to 3500 S/m, from 500 S/m to 2500 S/m, from 500 S/m to 1500 S/m, from 1500 S/m to 7500 S/m, from 1500 S/m to 6500 S/m, from 1500 S/m to 5500 S/m, from 1500 S/m to 4500 S/m, from 1500 S/m to 3500 S/m, from 1500 S/m to 2500 S/m, from 2500 S/m to 7500 S/m, from 2500 S/m to 6500 S/m, from 2500 S/m to 5500 S/m, from 2500 S/m to 4500 S/m, from 2500 S/m to 3500 S/m, from 3500 S/m to 7500 S/m, from 3500 S/m to 6500 S/m, from 3500 S/m to 5500 S/m, from 3500 S/m to 4500 S/m, from 4500 S/m to 7500 S/m, from 4500 S/m to 6500 S/m, from 4500 S/m to 5500 S/m, from 5500 S/m to 7500 S/m, from 5500 S/m to 6500 S/m, or from 6500 S/m to 7500 S/m at 20° C. Catalyst support materials having an electrical conductivity less than 50 S/m at 20° C. may not be capable of conducting an electrical current at a rate sufficient to heat the catalyst support material via joule heating.

The catalyst support material may have a specific surface area sufficient to enable reactants to efficiently pass through the catalyst support material to contact the catalytically active compound of the metathesis catalysts. Without being bound by any particular theory, it is believed that efficient passage of reactants through the catalyst support material may prevent limitation of the reaction kinetics by mass transfer of reactants through the catalyst support material. The catalyst support material may have a specific surface area of from 800 square meters per gram (m²/g) to 1000 m²/g, as determined by the Brunauer-Emmett-Teller (BET) method. For example, the catalyst support material may have a specific surface area of from 800 m²/g to 975 m²/g, from 800 m²/g to 950 m²/g, from 800 m²/g to 925 m²/g, from 800 m²/g to 900 m²/g, from 800 m²/g to 875 m²/g, from 800 m²/g to 850 m²/g, from 800 m²/g to 825 m²/g, from 825 m²/g to 1000 m²/g, from 825 m²/g to 975 m²/g, from 825 m²/g to 950 m²/g, from 825 m²/g to 925 m²/g, from 825 m²/g to 900 m²/g, from 825 m²/g to 875 m²/g, from 825 m²/g to 850 m²/g, from 850 m²/g to 1000 m²/g, from 850 m²/g to 975 m²/g, from 850 m²/g to 950 m²/g, from 850 m²/g to 925 m²/g, from 850 m²/g to 900 m²/g, from 850 m²/g to 875 m²/g, from 875 m²/g to 1000 m²/g, from 875 m²/g to 975 m²/g, from 875 m²/g to 950 m²/g, from 875 m²/g to 925 m²/g, from 875 m²/g to 900 m²/g, from 900 m²/g to 1000 m²/g, from 900 m²/g to 975 m²/g, from 900 m²/g to 950 m²/g, from 900 m²/g to 925 m²/g, from 925 m²/g to 1000 m²/g, from 925 m²/g to 975 m²/g, from 925 m²/g to 950 m²/g, from 950 m²/g to 1000 m²/g, from 950 m²/g to 975 m²/g, or from 975 m²/g to 1000 m²/g, as determined by the BET method.

The catalyst support material may have a pore volume sufficient to enable reactants to pass through the catalyst support material to contact the catalytically active compound of the metathesis catalysts. The pore volume of the catalyst support material may be a function of the pore size of the catalysts support material as well as the pore density of the catalyst support material. For a given average pore size, if the pore volume of the catalyst support material is too small, then the number of pathways through the catalyst support material to the catalytically active compound may be less, resulting in limiting the volumetric flow of reactants to the catalytically active compound of the metathesis catalyst, which may limit the reaction rate achievable by the system 100. The catalyst support material may have a pore volume of from 0.01 cubic centimeters per gram (cm³/g) to 1 cm³/g, as determined by single point adsorption (p/p₀=0.991). For example, the catalyst support material may have a pore volume of from 0.01 cm³/g to 0.8 cm³/g, from 0.01 cm³/g to 0.6 cm³/g, from 0.01 cm³/g to 0.4 cm³/g, from 0.01 cm³/g to 0.2 cm³/g, from 0.2 cm³/g to 1 cm³/g, from 0.2 cm³/g to 0.8 cm³/g, from 0.2 cm³/g to 0.6 cm³/g, from 0.2 cm³/g to 0.4 cm³/g, from 0.4 cm³/g to 1 cm³/g, from 0.4 cm³/g to 0.8 cm³/g, from 0.4 cm³/g to 0.6 cm³/g, from 0.6 cm³/g to 1 cm³/g, from 0.6 cm³/g to 0.8 cm³/g, or from 0.8 cm³/g to 1 cm³/g, as determined by single point adsorption (p/p₀=0.991).

The metathesis catalyst may comprise from 80 wt. % to 99 wt. % catalyst support material, based on the total weight of the metathesis catalyst. For example, metathesis catalyst 112 may comprise from 80 wt. % to 97 wt. %, from 80 wt. % to 95 wt. %, from 80 wt. % to 90 wt. %, from 80 wt. % to 85 wt. %, from 85 wt. % to 99 wt. %, from 85 wt. % to 97 wt. %, from 85 wt. % to 95 wt. %, from 85 wt. % to 90 wt. %, from 90 wt. % to 99 wt. %, from 90 wt. % to 97 wt. %, from 90 wt. % to 95 wt. %, from 95 wt. % to 99 wt. %, from 95 wt. % to 97 wt. %, or from 97 wt. % to 99 wt. % catalyst support material, based on the total weight of the metathesis catalyst.

As mentioned previously in the present disclosure, the metathesis catalysts may also comprise a catalytically active compound. The metathesis catalyst may generally comprise a catalytically active compound capable of promoting the self-metathesis and the cross-metathesis of butenes. The morphology, type, and amount of the catalytically active compound may determine the catalytic activity of the catalyst. The catalytically active compound may be a metal or metal oxide, such as one or more oxides of a metal from Groups 6-10 of the International Union of Pure and Applied Chemistry (IUPAC) Periodic Table, such as an oxide of molybdenum, rhenium, tungsten, manganese, titanium, cerium, or combination of these. For example, the catalytically active compound may be tungsten oxide. As noted previously in the present disclosure, the catalytically active compound of the metathesis catalyst may be supported by (that is, deposited on) the catalyst support material. The catalytically active compound of the metathesis catalyst may be dispersed throughout the catalyst support material, deposited on the surface of the catalysts support material, or combinations of these. Generally, at least a portion of the catalytically active compound may be accessible at the surfaces of the catalyst support material, such as at the outer and pore surfaces of the catalyst support material.

The metathesis catalyst may include a plurality of catalytically active sites. For example, the metathesis catalyst may have 1, 2, 3, 4, 5, 6, or more than 6, catalytically active sites. Theoretically, the number of different catalytically active sites that can be incorporated into the metathesis catalyst may be unlimited. However, the number of different catalytically active sites that can be included in the metathesis catalyst may be limited by the type of reactions that can be conducted simultaneously. The number of different catalytically active sites may also be limited by reactions that must be conducted sequentially. The number of different catalytically active sites may also be limited by catalyst poisoning considerations.

The metathesis catalyst may comprise the catalytically active compound in an amount sufficient to improve the reaction rate of the reactions catalyzed by the metathesis catalysts. The metathesis catalyst may comprise from 1 wt. % to 20 wt. % catalytically active compound, based on the total weight of the metathesis catalyst. For example, metathesis catalyst 112 may comprise from 1 wt. % to 16 wt. %, from 1 wt. % to 12 wt. %, from 1 wt. % to 8 wt. %, from 1 wt. % to 4 wt. %, from 4 wt. % to 20 wt. %, from 4 wt. % to 16 wt. %, from 4 wt. % to 12 wt. %, from 4 wt. % to 8 wt. %, from 8 wt. % to 20 wt. %, from 8 wt. % to 16 wt. %, from 8 wt. % to 12 wt. %, from 12 wt. % to 20 wt. %, from 12 wt. % to 16 wt. %, or from 16 wt. % to 20 wt. % catalytically active compound, based on the total weight of the metathesis catalyst.

It should be appreciated that various synthesis procedures may be utilized to prepare the metathesis catalyst of the present disclosure. For example, incipient wetness impregnation or hydrothermal synthesis may be suitable to prepare the metathesis catalyst. At least one example synthesis procedure, specifically an incipient wetness impregnation method, is described in the Examples section of the present disclosure.

The metathesis catalyst of the present disclosure may be incorporated into a system for metathesizing butene to produce propene and other olefins. Referring now to FIG. 1, a system 100 for producing a product stream comprising propene from a feedstock comprising butene is schematically depicted. The system 100 may generally include a metathesis reaction zone 110. Optionally, the system 100 may further include one or more additional reaction zones, such as isomerization reaction zones, additional metathesis reaction zones, or cracking reaction zones, which are not depicted. Generally, the metathesis reaction zones, including the metathesis reaction zone 110, may be positioned downstream of the isomerization reaction zones, and the cracking reaction zones may be positioned downstream of the metathesis reaction zones. As depicted in FIG. 1, the system 100 may comprise the metathesis reaction zone 110 disposed within a reactor 120. While not depicted, it should be understood that any additional reaction zones may also be disposed within the reactor 120, such as in a series of catalyst beds. Alternatively, each reaction zone, including the metathesis reaction zone 110, may be disposed within independent reactors arranged in series. For example, the system 100 may comprise three reactors arranged in series where the first reactor comprises an isomerization reaction zone, the second reactor comprises a metathesis reaction zone, and the third reactor comprises a cracking reaction zone. The isomerization effluent of the first reactor may enter the second reactor as an inlet stream, the metathesis effluent of the second reactor may enter the third reactor as an inlet stream, and the cracking effluent of the third reactor may exit the system 100 as a product stream.

Referring still to FIG. 1, a feedstock 130 may be introduced to the system 100 by entering the reactor 120 as an inlet stream. As mentioned previously in the present disclosure, the feedstock 130 may generally comprise butene, such as 1-butene, trans-2-butene, cis-2-butene, or combinations of one or more of these isomers. The feedstock 130 may comprise from 0 (zero) weight percent (wt. %) to 100 wt. % 1-butene, based on the total weight of the feedstock 130. For example, the feedstock 130 may comprise from 0 wt. % to 75 wt. %, from 0 wt. % to 50 wt. %, from 0 wt. % to 25 wt. %, from 25 wt. % to 100 wt. %, from 25 wt. % to 75 wt. %, from 25 wt. % to 50 wt. %, from 50 wt. % to 100 wt. %, from 50 wt. % to 75 wt. %, or from 75 wt. % to 100 wt. % 1-butene, based on the total weight of the feedstock 130. The feedstock 130 may comprise from 0 weight percent (wt. %) to 100 wt. % 2-butene, based on the total weight of the feedstock 130. For example, the feedstock 130 may comprise from 0 wt. % to 75 wt. %, from 0 wt. % to 50 wt. %, from 0 wt. % to 25 wt. %, from 25 wt. % to 100 wt. %, from 25 wt. % to 75 wt. %, from 25 wt. % to 50 wt. %, from 50 wt. % to 100 wt. %, from 50 wt. % to 75 wt. %, or from 75 wt. % to 100 wt. % 2-butene, based on the total weight of the feedstock 130. Optionally, the feedstock may be substantially free of ethylene. As used throughout the present disclosure, the term “substantially free” of a material or component may refer to a particular process stream, such as feedstock 130, catalyst composition, or reaction zone that comprises less than 1 wt. % of the material or component. For example, the feedstock 130, which may be substantially free of ethylene, may comprise less than 1 wt. %, less than 0.8 wt. %, less than 0.6 wt. %, less than 0.4 wt. %,less than 0.2 wt. %, or less than 0.1 wt. % ethylene, based on the total weight of the feedstock 130.

Accordingly, the feedstock 130 may comprise various sources comprising sufficient amounts of butene for the production of propene, such as a raffinate stream. As used throughout the present disclosure, the term “raffinate” refers to a residue stream resulting from the processing of various hydrocarbon streams, such as the residue stream resulting from a naphtha cracking process or a gas cracking process. Raffinate streams generally comprise n-butane, 1-butene, 2-butene, isobutene, 1,3-butadiene, isobutene, or combinations of these, as primary components. That is, raffinate streams generally comprise greater than or equal to 99 wt. % n-butane, 1-butene, 2-butene, isobutene, 1,3-butadiene, isobutene, or combinations of these, based on the total weight of the raffinate stream. Raffinate streams may also be further refined to produce raffinate-1 streams, raffinate-2 streams, raffinate-3 streams, or raffinate-4 streams. Raffinate-1 streams may be produced by the separation of 1,3-butadiene from the separation of a raffinate stream, and generally comprises greater than 50 wt. % isobutene, 2-butene, or combinations of these, based on the total weight of the raffinate-1 stream. Raffinate-2 streams may be produced by the separation of 1,3-butadiene and isobutene from a raffinate stream, and generally comprises greater than 50 wt. % 2-butene, 1-butene, n-butane, or combinations of one or more of these, based on the total weight of the raffinate-2 stream. Raffinate-3 streams may be produced by the separation of 1,3-butadiene, isobutene, and 1-butene from a raffinate stream, and generally comprises greater than 50 wt. % 2-butene, n-butane, unseparated 1-butene, or combinations of one or more of these, based on the total weight of the raffinate-3 stream. Raffinate-4 streams may be produced by the separation of 1,3-butadiene, isobutene, 1-butene, and 2-butene from a raffinate stream, and generally comprises greater than 50 wt. % n-butene, based on the total weight of the raffinate-4 stream. Accordingly, the feedstock 130 may comprise a raffinate stream, a raffinate-1 stream, a raffinate-2 stream, a raffinate-3 stream, or combinations of one or more of these streams.

Referring still to FIG. 1, the feedstock 130 may be introduced to the system 100 by entering the reactor 120 as an inlet stream and a metathesis reaction effluent 140 may be passed out of the system 100 by exiting the reactor 120 as a product stream. Accordingly, the feedstock 130 may enter the reactor 120, pass through the metathesis reaction zone 110, and exit the reactor 120 as the metathesis reaction effluent 140. In particular, the feedstock 130 may enter the reactor 120, pass through the metathesis reaction zone 110, which comprises a metathesis catalyst 112, and exit the reactor 120 as the metathesis reaction effluent 140. It should be understood that metathesis catalyst 112 may be in accordance with the metathesis catalysts previously described in the present disclosure.

Referring still to FIG. 1, the metathesis catalyst 112 may be heated to an operational temperature sufficient to promote the production of propene from a feedstock comprising butene, such as, for example, feedstock 130. The operational temperature may be from 400° C. to 600° C. For example, the operational temperature may be from 400° C. to 575° C., from 400° C. to 550° C., from 400° C. to 525° C., from 400° C. to 500° C., from 400° C. to 475° C., from 400° C. to 450° C., from 400° C. to 425° C., from 425° C. to 600° C., from 425° C. to 575° C., from 425° C. to 550° C., from 425° C. to 525° C., from 425° C. to 500° C., from 425° C. to 475° C., from 425° C. to 450° C., from 450° C. to 600° C., from 450° C. to 575° C., from 450° C. to 550° C., from 450° C. to 525° C., from 450° C. to 500° C., from 450° C. to 475° C., from 475° C. to 600° C., from 475° C. to 575° C., from 475° C. to 550° C., from 475° C. to 525° C., from 475° C. to 500° C., from 500° C. to 600° C., from 500° C. to 575° C., from 500° C. to 550° C., from 500° C. to 525° C., from 525° C. to 600° C., from 525° C. to 575° C., from 525° C. to 550° C., from 550° C. to 600° C., from 550° C. to 575° C., or from 575° C. to 600° C. The metathesis catalyst 112 may promote the production of propene from a feedstock comprising butene when heated to an operational temperature within these ranges. In particular, the metathesis catalyst 112 may promote both self-metathesis of butene, such as the self-metathesis of 2-butene to 1-butene, and the cross-metathesis of butene, such as the cross-metathesis of 2-butene and 1-butene to produce propene, when heated to an operational temperature within these ranges. Without being bound by any particular theory, it is believed that at temperatures below 400° C., the metathesis catalysts 112 may fail to promote the self-metathesis of butene. As a result, the yield of propene may be reduced, particularly when feedstocks comprising disproportionate amounts of 1-butene or 2-butene are used.

Conventionally, metathesis catalysts are heated via a furnace utilizing stainless steel or ceramic fiber heating elements. Conventional furnaces can consume significant amounts of power in order to be heated to a desired temperature. For example, a 22.7-kilogram furnace utilizing a stainless steel heating element may require approximately 2150 watts to be heated to 500° C. within 1 hour, and may require approximately 8600 watts to be heated to 500° C. within 15 minutes. Similarly, a 5-kilogram furnace utilizing a stainless steel heating element may require approximately 473 watts to be heated to 500° C. within 1 hour, and may require approximately 1892 watts to be heated to 500° C. within 15 minutes. As mentioned discussed, the metathesis catalyst of the present disclosure having an electrically conductive catalyst support material may enable joule heating to be used to heat the metathesis catalysts. As used in the present disclosure, the term “joule heating,” also known as ohmic or resistive heating, may refer to the process of passing an electric current through a material to produce heat. For example, metathesis catalyst 112 may be joule heated to an operational temperature by passing an electric current through the catalyst support material from a power source via two or more electrodes. Joule heating may not be suitable when a metathesis catalyst comprises a catalysts support material that does not have a sufficiently high electrical or thermal conductivity, as discussed previously in the present disclosure. For example, joule heating may not be suitable for use with metathesis catalysts that comprise a silica catalyst support material as silica acts as an insulator with poor electrical and thermal conductivity.

When joule heating the metathesis catalyst 112, an electric current may be applied to at least the catalyst support material of the metathesis catalysts 112 in an amount sufficient to heat the metathesis catalysts 112 to an operational temperature. When joule heating the metathesis catalyst 112, an electric current may be applied to at least the catalyst support material of the metathesis catalysts 112 such that from 20 watts to 40 watts are delivered to the catalyst support material. For example, an electric current may be applied to at least the catalyst support material of the metathesis catalysts 112 such that from 20 watts to 38 watts, from 20 watts to 36 watts, from 20 watts to 34 watts, from 20 watts to 32 watts, from 20 watts to 30 watts, from 20 watts to 28 watts, from 20 watts to 26 watts, from 20 watts to 24 watts, from 20 watts to 22 watts, from 22 watts to 40 watts, from 22 watts to 38 watts, from 22 watts to 36 watts, from 22 watts to 34 watts, from 22 watts to 32 watts, from 22 watts to 30 watts, from 22 watts to 28 watts, from 22 watts to 26 watts, from 22 watts to 24 watts, from 24 watts to 40 watts, from 24 watts to 38 watts, from 24 watts to 36 watts, from 24 watts to 34 watts, from 24 watts to 32 watts, from 24 watts to 30 watts, from 24 watts to 28 watts, from 24 watts to 26 watts, from 26 watts to 40 watts, from 26 watts to 38 watts, from 26 watts to 36 watts, from 26 watts to 34 watts, from 26 watts to 32 watts, from 26 watts to 30 watts, from 26 watts to 28 watts, from 28 watts to 40 watts, from 28 watts to 38 watts, from 28 watts to 36 watts, from 28 watts to 34 watts, from 28 watts to 32 watts, from 28 watts to 30 watts, from 30 watts to 40 watts, from 30 watts to 38 watts, from 30 watts to 36 watts, from 30 watts to 34 watts, from 30 watts to 32 watts, from 32 watts to 40 watts, from 32 watts to 38 watts, from 32 watts to 36 watts, from 32 watts to 34 watts, from 34 watts to 40 watts, from 34 watts to 38 watts, from 34 watts to 36 watts, from 36 watts to 40 watts, from 36 watts to 38 watts, or from 38 watts to 40 watts are delivered to the catalyst support material. Such an application of electric current to at least the catalysts support material of the metathesis catalysts 112 may be sufficient to heat the metathesis catalysts 112 to an operational temperature. Moreover, as noted previously in the present disclosure, such an application of electric current directly to the metathesis catalysts may require significantly less power compared to conventional methods of heating a catalysts via a furnace.

Referring now to FIG. 2, a fluid-solid separator 150 may be disposed downstream of the metathesis reaction zone 110, upstream of the metathesis reaction zone 110, or combinations of these. As used in the present disclosure, the term “fluid-solid separator” may refer to a fluid permeable barrier adjacent to or between catalyst beds that substantially prevents solid catalyst particles in one catalyst bed from migrating from or to an adjacent catalyst bed, while allowing for reactants and products to move through the separator. The fluid-solid separator 150 may be chemically inert and generally makes no contribution to the reaction chemistry. Inserting the fluid/solid separator 150 adjacent to the metathesis reaction zone 110 may maintain the metathesis catalyst 112 in the metathesis reaction zone 110, and prevent migration of different catalysts between any additional reaction zones, which may lead to increased production of undesired by-products and decreased yield.

Referring again to FIG. 1, various operating conditions are contemplated for contacting the feedstock 130 with the metathesis catalysts 112. For example, the reactor 120 may be operated and maintained at a gas space hour velocity (GSHV) of from 10 per hour (h⁻¹) to 10,000 h⁻¹. Accordingly, the reactor 120 may be operated and maintained at a gas space hour velocity of from 10 h⁻¹ to 5000 h⁻¹, from 10 h⁻¹ to 2500 h⁻¹, from 10 h⁻¹ to 1250 h⁻¹, from 10 h⁻¹ to 625 h⁻¹, from 625 h⁻¹ to 10,000 h⁻¹, from 625 h⁻¹ to 5000 h⁻¹, from 625 h⁻¹ to 2500 h⁻¹, from 625 h⁻¹ to 1250 h⁻¹, from 1250 h⁻¹ to 10,000 h⁻¹, from 1250 h⁻¹ to 5000 h⁻¹, from 1250 h⁻¹ to 2500 h⁻¹, from 2500 h⁻¹ to 10,000 h⁻¹, from 2500 h⁻¹ to 5000 h⁻¹, or from 5000 h⁻¹ to 10,000 h⁻¹. Furthermore, the reactor 120 may be operated and maintained at a pressure of from 1 bar to 30 bars. For example, the reactor 120 may be operated and maintained at a pressure of from 1 bar to 25 bars, from 1 bar to 20 bars, from 1 bar to 15 bars, from 1 bar to 10 bars, from 1 bar to 5 bars, from 5 bars to 30 bars, from 5 bars to 25 bars, from 5 bars to 20 bars, from 5 bars to 15 bars, from 5 bars to 10 bars, from 10 bars to 30 bars, from 10 bars to 25 bars, from 10 bars to 20 bars, from 10 bars to 15 bars, from 15 bars to 30 bars, from 15 bars to 25 bars, from 15 bars to 20 bars, from 20 bars to 30 bars, from 20 bars to 25 bars, or from 25 bars to 30 bars. Alternatively, the reactor 120 may be operated and maintained at atmospheric pressure.

Optionally, prior to the introduction of the feedstock 130 to the system 100, the catalysts may be pretreated. For example, the metathesis catalyst 112 may be pretreated by passing a heated gas stream through the system 100 for a pretreatment period. The gas stream may include one or more of an oxygen-containing gas, nitrogen gas (N₂), carbon monoxide (CO), hydrogen gas (H₂), a hydrocarbon gas, air, other inert gas, or combinations of these gases. The temperature of the heated gas stream may be from 250° C. to 700° C., from 250° C. to 600° C., from 250° C. to 500° C., from 250° C. to 400° C., from 250° C. to 300° C., from 300° C. to 700° C., from 300° C. to 600° C., from 300° C. to 500° C., from 300° C. to 400° C., from 400° C. to 700° C., from 400° C. to 600° C., from 400° C. to 500° C., from 500° C. to 700° C., from 500° C. to 600° C., or from 600° C. to 700° C. The pretreatment period may be from 1 minute to 30 hours, from 1 minute to 20 hours, from 1 minute to 10 hours, from 1 minute to 5 hours, from 1 minute to 1 hour, from 1 minute to 30 minutes, from 30 minutes to 30 hours, from 30 minutes to 20 hours, from 30 minutes to 20 hours, from 30 minutes to 10 hours, from 30 minutes to 5 hours, from 30 minutes to 1 hour, from 1 hour to 30 hours, from 1 hour to 20 hours, from 1 hour to 10 hours, from 1 hour to 5 hours, from 5 hours to 30 hours, from 5 hours to 20 hours, from 5 hours to 10 hours, from 10 hours to 30 hours, from 10 hours to 20 hours, or from 20 hours to 30 hours. For example, the metathesis catalyst 112 may be pretreated with nitrogen gas at a temperature of 550° C. for a pretreatment period of from 1 hour to 5 hours before introducing the feedstock 130 to the system 100.

As noted previously in the present disclosure, during operation of system 100 the feedstock 130 may be contacted with the metathesis catalyst 112 in the metathesis reaction zone 110 to produce a metathesis reaction effluent 140 that comprises propene, as well as other alkanes and alkenes, such as C₅+ olefins, for example. The metathesis reaction effluent 140 may also include unreacted butenes, such as 1-butene, cis-2-butene, trans-2-butene, or combinations of these. After exiting the metathesis reaction zone 110, the metathesis reaction effluent 140 may be passed to one or more additional reaction zone, such as a second metathesis reaction zone or a cracking reaction zone, or may be exit the system 100 as a product stream.

Accordingly, methods of producing propene from a feedstock comprising butene may generally comprise heating a metathesis catalysts to an operational temperature. The metathesis catalysts may be in accordance with the metathesis catalysts described previously in the present disclosure. For example, the metathesis catalysts may comprise from 80 wt. % to 99 wt. % of a catalyst support material comprising carbon, and from 1 wt. % to 20 wt. % of a catalytically active compound comprising tungsten. The heating of the metathesis catalysts may also be in accordance with the heating previously described in the present disclosure. For example, the metathesis catalysts may be heated by joule heating the catalysts support material. The methods may further comprise contacting the feedstock with the metathesis catalyst. The feedstock may be contacted with the metathesis catalyst via the system previously described in the present disclosure. The contacting of the feedstock with the metathesis catalyst at the operational temperature may result in at least a portion of the butene in the feedstock to undergo a metathesis reaction and produce propene.

### EXAMPLES

The various embodiments of metathesis catalysts and methods of producing propene from a feedstock comprising butene will be further clarified by the following examples. The examples are illustrative in nature, and should not be understood to limit the subject matter of the present disclosure.

### Example 1

### Preparation of Metathesis Catalyst Comprising Tungsten Oxide and Activated Carbon Beads

For Example 1, a metathesis catalyst was prepared using tungsten oxide as the catalytically active compound and activated carbon beads as the catalyst support material by using an incipient wetness impregnation technique. A catalyst precursor mixture was prepared by adding 0.5896 grams of ammonium metatungstate hydrate, commercially available from Sigma Aldrich, and 5 grams of activated carbon beads, commercially available as MatrixCarbon® from Seachem, to 20 milliliters of deionized water. The catalyst precursor mixture was transferred to a rotary evaporator, which was rotated at 140 rotation per minute (rpm) and operated under a vacuum of 80 millibar (mbar) and at a temperature of 80° C. The resulting catalyst precursor slurry was then dried overnight in an oven at 80° C.

### Example 2

### Preparation of Metathesis Catalyst Comprising Tungsten Oxide and Activated Carbon Powder

For Example 2, a metathesis catalyst was prepared using tungsten oxide as the catalytically active compound and activated carbon powder as the catalyst support material by using an incipient wetness impregnation technique. A catalyst precursor mixture was prepared by adding 0.5896 grams of ammonium metatungstate hydrate, commercially available from Sigma Aldrich, and 5 grams of activated carbon powder, commercially available as Activated Charcoal from Sigma Aldrich, to 20 milliliters of deionized water. The catalyst precursor mixture was transferred to a rotary evaporator, which was rotated at 140 rpm and operated under a vacuum of 80 mbar and at a temperature of 80° C. The resulting catalyst precursor slurry was then dried overnight in an oven at 80° C.

### Comparative Example 3

### Preparation of Silica Catalyst Support Material

For Comparative Example 3, a silica catalyst support material was prepared by calcining silica, commercially available as CARiACT® Q-10 from Fuji Silysia Chemical. The silica was calcined under air in a calcination oven, which was heated at a ramping rate of 3 degrees Celsius per minute (° C./min) until a temperature of 200° C. was achieved. The silica was then maintained in the calcination oven at a temperature of 200° C. for 3 hours. The ramping rate of 3° C./min was then resumed until the calcination oven achieved a temperature of 575° C. The silica was then maintained in the calcination oven at a temperature of 575° C. for 5 hours. Following calcination, the resulting silica catalyst support material was maintained in the calcination oven and allowed to slowly cool to room temperature.

### Comparative Example 4

### Preparation of Metathesis Catalyst Comprising Tungsten Oxide and Silica

For Comparative Example 4, a metathesis catalyst was prepared using tungsten oxide as the catalytically active compound and silica as the catalyst support material. A catalyst precursor mixture was prepared by adding 5.896 grams of ammonium metatungstate hydrate, commercially available from Sigma Aldrich, and 20 grams of the silica catalyst support material of Comparative Example 1 to 200 milliliters of deionized (DI) water. The catalyst precursor mixture was mixed for 30 minutes at 400 rotations per minute (rpm). After mixing for 30 minutes, the catalyst precursor mixture was transferred to a rotary evaporator, which was operated at a temperature of 80° C. The resulting catalyst precursor slurry was then dried overnight in an oven at 80° C. After drying overnight, the resulting powder was crushed and calcined under air in a calcination oven, which was heated at a ramping rate of 1° C./min until a temperature of 250° C. was achieved. The powder was then maintained in the calcination oven at a temperature of 250° C. for 2 hours. Then the heating of the calcination oven was resumed at a ramping rate of 3° C./min a temperature of 550° C. was achieved. The powder was then maintained in the calcination oven at a temperature of 550° C. for 8 hours. Following calcination, the resulting metathesis catalyst was maintained in the calcination oven and allowed to slowly cool to room temperature.

### Example 5

### Evaluation of the Joule Heating of the Metathesis Catalyst of Example 1

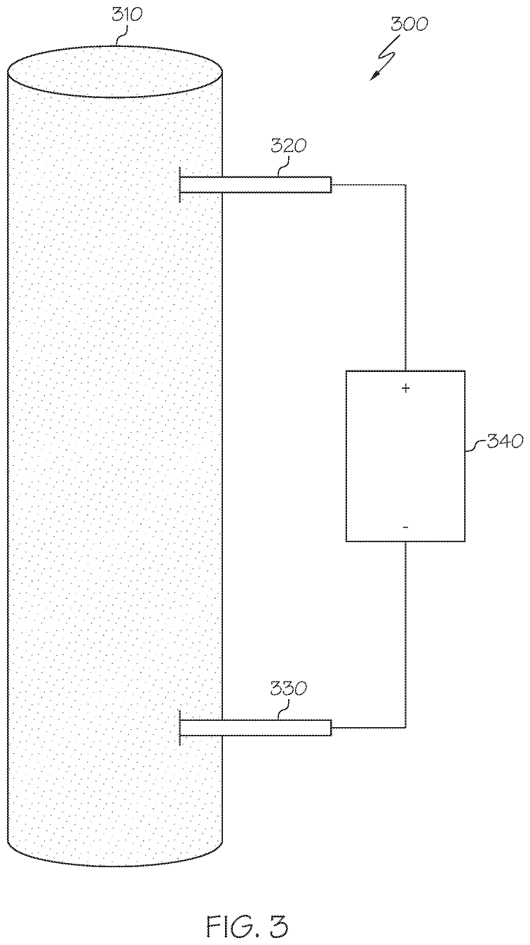

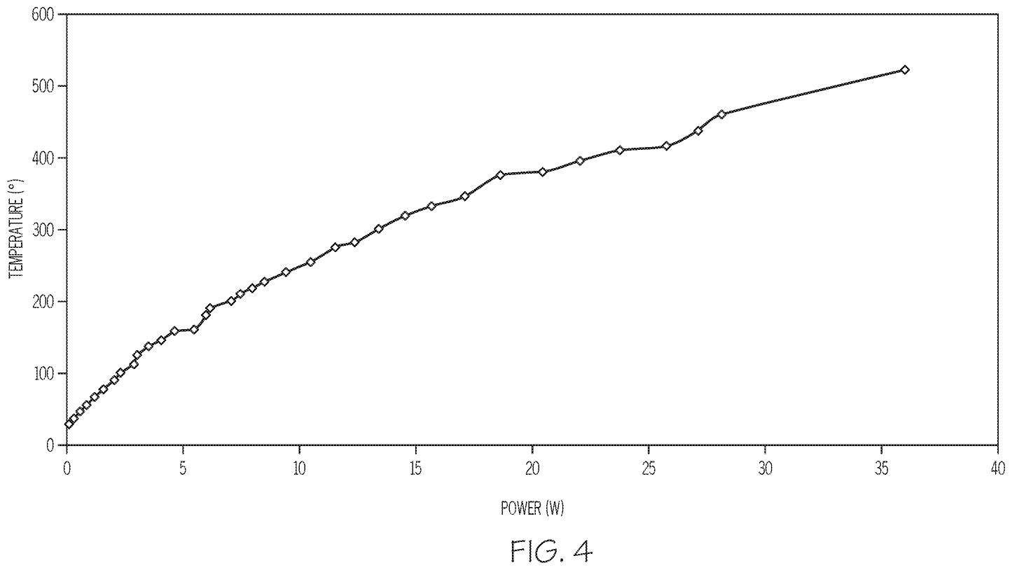

For Example 5, the metathesis catalyst of Example 1 was evaluated to determine the amount of power necessary to joule heat the metathesis catalyst to a desired temperature. In order to determine the amount of power necessary to joule heat the metathesis catalyst of Example 1, 100 milligrams of the metathesis catalyst was transferred to an experimental apparatus 300, as depicted in FIG. 3. As depicted in FIG. 3, the experimental apparatus 300 generally comprised a quartz tube 310 and two electrodes, cathode 320 and anode 330, which were connected to a power supply 340, commercially available from Thurlby Thandar Instruments. After transferring 100 milligrams of the metathesis catalyst was transferred to the quartz tube 310, cathode 320 and anode 310 were inserted into the metathesis catalyst. An electric current was then supplied to the metathesis catalyst from the power supply 340 such that the power applied to metathesis catalyst gradually increased from 0 watts to 35 watts, while the temperature of the metathesis catalyst was measured using a thermocouple. FIG. 4 graphically depicts the results as the temperature of the metathesis catalyst (y-axis) as a function of power applied to the metathesis catalyst (x-axis).

As shown by FIG. 4, relatively low amounts of power may be required to effectively joule heat the metathesis catalyst of Example 1. That is, approximately 35 watts were sufficient to effectively heat 100 milligrams of the metathesis catalyst of Example 1 to an operation temperature of 500° C. In comparison, conventional catalysts, which are typically heated using a furnace, may have much greater power requirements. For example, a large (22.7 kilogram) stainless steel furnace used to heat a conventional silica supported catalyst may require greater than 2000 watts to be heated to 500° C. in 1 hour, or even greater than 8000 watts to be heated to 500° C. in 15 minutes. Similarly, a smaller (5 kilogram) stainless steel furnace used to heat a conventional silica supported catalyst may require greater than 400 watts to be heated to 500° C. in 1 hour, or even greater than 1800 watts to be heated to 500° C. in 15 minutes. As such, the utilization of joule heating may drastically decrease the energy requirements and efficiency of reactor system.

### Example 6

### Evaluation of Catalyst Support Materials

In Example 6, crystallographic structures of a silica catalyst support material and an activated carbon support material were obtained from the measured XRD profiles of the catalyst support materials. Before measurement of the XRD profiles, samples of silica, commercially available as CARiACT® Q-10 from Fuji Silysia Chemical, and activated carbon beads, commercially available as MatrixCarbon® from Seachem, were calcined under air in a calcination oven, which was heated at a ramping rate of 1° C./min until a temperature of 200° C. was achieved. The silica and activated carbon samples were then maintained in the calcination oven at a temperature of 200° C. for 3 hours. Then the heating of the calcination oven was resumed at a ramping rate of 3° C./min a temperature of 550° C. was achieved. The silica and activated carbon samples were then maintained in the calcination oven at a temperature of 550° C. for 5 hours. Following calcination, the resulting catalyst support materials were maintained in the calcination oven and allowed to slowly cool to room temperature.

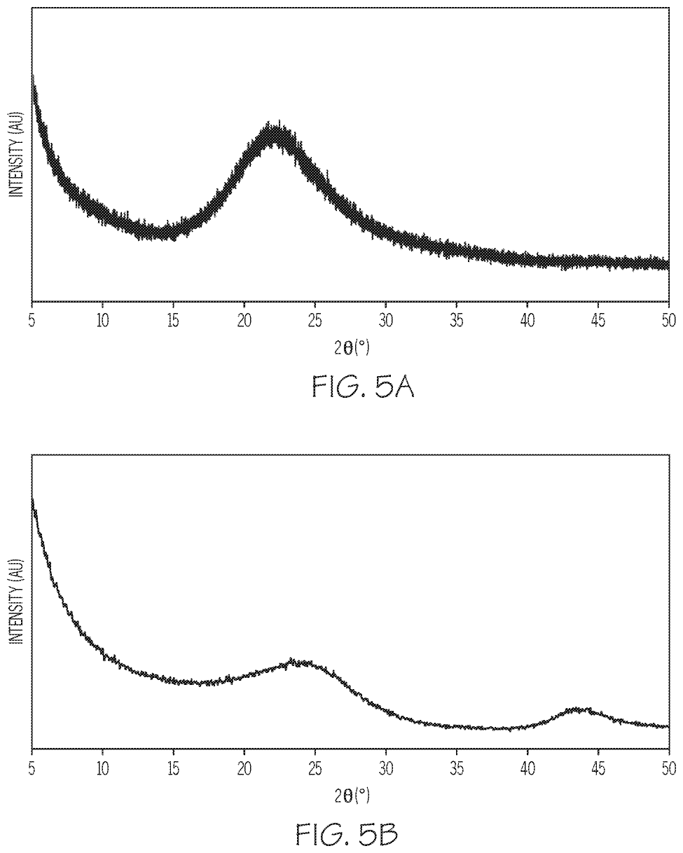

FIG. 5A graphically depicts the XRD profile of the silica catalyst support material and FIG. 5B graphically depicts the XRD profile of the activated carbon support material. As depicted in FIGS. 5A and 5B, both the silica catalyst support material and the activated carbon support material have amorphous structures. This is indicated by the characteristic diffraction peaks between 18 degrees)(°) and 30° present in both XRD profiles, as well as the broad diffraction peak between 40° and 50° in the XRD profile of the activated carbon catalyst support material. Without being bound by any particular theory, it is believed that such an amorphous structure may remain intact after the deposition of a catalytically active material on the surface and allow for the catalytically active material to be deposited in an optimum ratio. In contrast, a crystalline structure may result in an excess of catalytically active material and a reduction of catalytic activity.

### Comparative Example 7

### Evaluation of the Metathesis Catalyst of Comparative Example 4

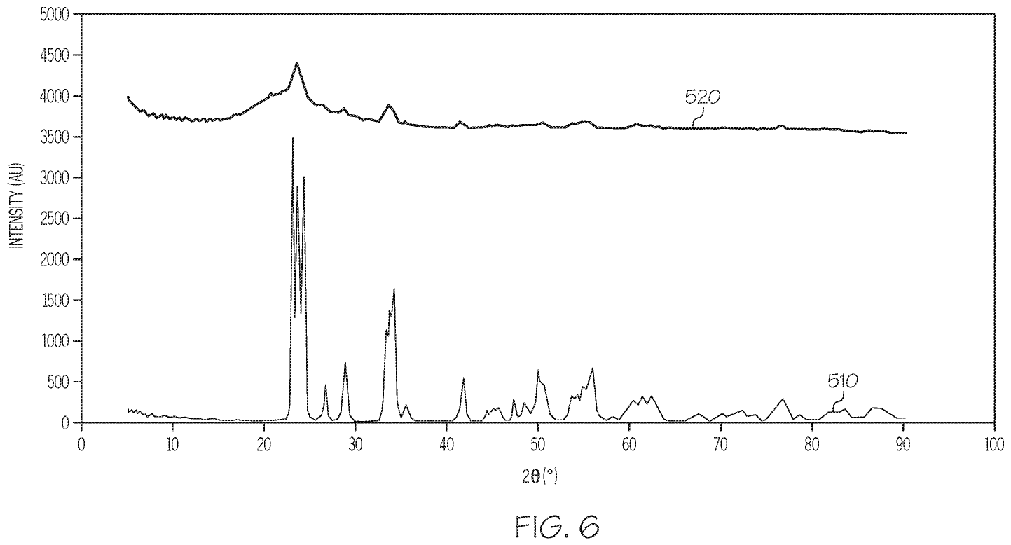

In Comparative Example 7, crystallographic structures of the metathesis catalyst of Comparative Example 4 was obtained from the measured XRD profile of the metathesis catalyst. FIG. 6 graphically depicts the XRD profile of the metathesis catalyst of Comparative Example 4 (520) as well as the XRD profile of tungsten oxide (510). As depicted in FIG. 6, the diffraction peaks of the XRD profile of tungsten oxide in in line with the standard XRD profile of tungsten oxide, as determined by the JCPDS-International Centre for Diffraction Data. Moreover, a comparison of the two XRD profiles allows the characteristic diffraction peaks of tungsten oxide to be clearly identified emerging from the broader characteristic diffraction peaks of silica. This confirms that the metathesis catalyst of Comparative Example 4 includes both tungsten oxide and silica.

### Example 8

### Evaluation of the Metathesis Catalysts of Examples 1 and 2

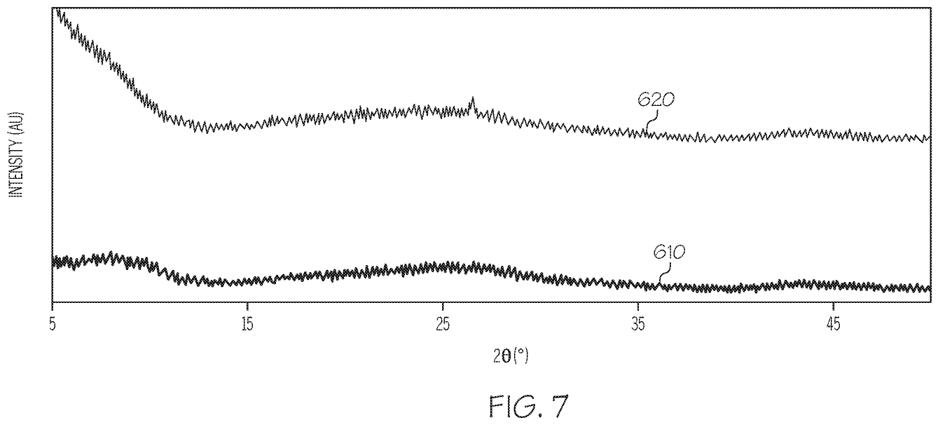

In Example 8, crystallographic structures of the metathesis catalysts of Examples 1 and 2 were obtained from the measured XRD profiles of the metathesis catalysts. FIG. 7 graphically depicts the XRD profile of the metathesis catalyst of Example 1 (620) as well as the XRD profile of the metathesis catalyst of Example 2 (610). As depicted in FIG. 7, the characteristic diffraction peaks of activated carbon, such as those described in Example 6, become broader and less defined after impregnation with tungsten oxide. While the XRD profiles confirm that the metathesis catalysts of Example 1 and 2 include both tungsten oxide and activated carbon, the XRD profiles also confirm that the characteristic peaks of tungsten oxide may be less visible when compared to the XRD profile of, for example, Comparative Example 7 due to overlap with the broad diffraction peaks of activated carbon.

### Example 9

### Evaluation of the Metathesis Catalysts of Examples 1 and 2 and Comparative Example 2

In Example 9, the metathesis catalysts of Examples 1 and 2 and Comparative Example 3 were evaluated for activity and selectivity in converting 2-butene to propene in a fixed-bed continuous-flow reactor, commercially available from Autoclave Engineers Ltd., at atmospheric pressure. Each reactor tube was first packed with a layer of quartz wool, followed by a layer of silicon carbide, followed by a second layer of quartz wool. A fixed amount of the metathesis catalysts were then packed into the reactor tubes, followed by a final layer of quartz wool. Each metathesis catalysts was then pretreated under nitrogen gas (N₂) at 550° C. and a flow rate of 25 standard cubic centimeters per minute (sccm) for 60 minutes. After pretreatment, the temperature of the nitrogen gas was lowered to 450° C. and a feedstock of 2-butene was introduced to the reactors. Each reactor was maintained at a gas hourly space velocity (GHSV) of 900 per hour (h⁻¹) and quantitative analysis of the products for each reactor was performed using a gas chromatograph (commercially available as Agilent GC-7890B). The products of each reactor were analyzed four times and the average results for each reactor are summarized in Table 1.

TABLE 1

| | 2-Butene | Propene | Propene |
|---|---|---|---|
| Catalyst | Conversion % | Yield % | Selectivity % |
| Example 1 | 39.31 | 12.5 | 31.81 |
| Example 2 | 38.14 | 11.1 | 29.10 |
| Comparative Example 3 | 60 | 19.9 | 33.16 |

As shown by Table 1, the metathesis catalyst of Comparative Example 3 had a significantly greater conversion rate of 2-butene, but only a slightly greater yield of propene, and the selectivity for propene was similar for all three metathesis catalysts. While this indicates that the metathesis catalyst of Comparative Example 3 may provide greater catalytic activity with regards to the conversion of 2-butene through metathesis, it also indicates that the metathesis catalysts of Examples 1 and 2 also have catalytic activity suitable to promote the metathesis of 2-butene to propene. This may be shown, at least in part, by the similar propene selectivity and yields achieved by each metathesis catalyst. Moreover, as noted previously in the present disclosure, the metathesis catalysts of Examples 1 and 2 comprise an activated carbon catalyst support material and, as a result, are capable of being heated much more efficiently than the metathesis catalyst of Comparative Example 3, which comprises a silica catalyst support material.

It will be apparent to those skilled in the art that various modifications and variations can be made without departing from the spirit or scope of the disclosure. Since modifications, combinations, sub-combinations and variations of the disclosed embodiments incorporating the spirit and substance of the disclosure may occur to persons skilled in the art, the scope of the disclosure should be construed to include everything within the scope of the appended claims their equivalents.

In a first aspect of the present disclosure, a metathesis catalyst may comprise 80 weight percent to 99 weight percent catalyst support material, based on the total weight of the metathesis catalyst, where the catalyst support material comprises carbon; and 1 weight percent to 20 weight percent catalytically active compound, based on the total weight of the metathesis catalyst, where the catalytically active compound comprises tungsten and is supported by the catalyst support material.

A second aspect of the present disclosure may comprise the first aspect where the catalyst support material has a specific surface area of from 800 square meters per gram to 1000 square meters per gram.

A third aspect of the present disclosure may comprise either of the first or second aspects where the catalyst support material has an average particle size of from 1 millimeter to 5 millimeters.

A fourth aspect of the present disclosure may comprise any of the first through third aspects where the catalyst support material has a pore volume of from 0.01 cubic centimeters per gram to 1 cubic centimeter per gram.

A fifth aspect of the present disclosure may comprise any of the first through fourth aspects where the catalyst support material comprises activated carbon.

A sixth aspect of the present disclosure may comprise any of the first through fifth aspects where the catalytically active compound comprises tungsten oxide.

In a seventh aspect of the present disclosure, a method of producing propene from a feedstock comprising butene may comprise heating a metathesis catalyst to an operational temperature, where the metathesis catalyst comprises: 80 weight percent to 99 weight percent catalyst support material, based on the total weight of the metathesis catalyst, where the catalyst support material comprises carbon; and 1 weight percent to 20 weight percent catalytically active compound, based on the total weight of the metathesis catalyst, where the catalytically active compound comprises tungsten and is supported by the catalyst support material; and contacting the feedstock with the metathesis catalyst, where the contacting of the feedstock with the metathesis catalyst at the operational temperature results in at least a portion of the butene in the feedstock to undergo a metathesis reaction and produce propene.

An eighth aspect of the present disclosure may comprise the seventh aspect where heating the metathesis catalyst comprises joule heating at least the catalyst support material.

A ninth aspect of the present disclosure may comprise the eighth aspect where joule heating at least the catalyst support material comprises applying an electric current to at least the catalyst support material such that from 20 watts to 40 watts are delivered to the catalyst support material.

A tenth aspect of the present disclosure may comprise any of the seventh through ninth aspects where the operational temperature is from 400 degrees Celsius to 600 degrees Celsius.

An eleventh aspect of the present disclosure may comprise any of the seventh through tenth aspects where the catalyst support material has a specific surface area of from 800 square meters per gram to 1000 square meters per gram.

A twelfth aspect of the present disclosure may comprise any of the seventh through eleventh aspects where the catalyst support material has an average particle size of from 1 millimeter to 5 millimeters.

A thirteenth aspect of the present disclosure may comprise any of the seventh through twelfth aspects where the catalyst support material has a pore volume of from 0.01 cubic centimeters per gram to 1 cubic centimeter per gram.

A fourteenth aspect of the present disclosure may comprise any of the seventh through thirteenth aspects where the catalyst support material comprises activated carbon.

A fifteenth aspect of the present disclosure may comprise any of the seventh through fourteenth aspects where the catalytically active compound comprises tungsten oxide.

In a sixteenth aspect of the present disclosure, a method of producing propene from a feedstock comprising butene may comprise joule heating a metathesis catalyst to an operational temperature; and contacting the feedstock with the metathesis catalyst, where the contacting of the feedstock with the metathesis catalyst at the operational temperature results in at least a portion of the butene in the feedstock to undergo a metathesis reaction and produce propene.

A seventeenth aspect of the present disclosure may comprise the sixteenth aspect where joule heating at least the catalyst support material comprises applying an electric current to at least the catalyst support material such that from 20 watts to 40 watts are delivered to the catalyst support material.

An eighteenth aspect of the present disclosure may comprise either of the sixteenth or seventeenth aspects where the operational temperature is from 400 degrees Celsius to 600 degrees Celsius.

A nineteenth aspect of the present disclosure may comprise any of the sixteenth through seventeenth aspects where the metathesis catalyst comprises 80 weight percent to 99 weight percent catalyst support material, based on the total weight of the metathesis catalyst, where the catalyst support material comprises carbon; and 1 weight percent to 20 weight percent catalytically active compound, based on the total weight of the metathesis catalyst, where the catalytically active compound comprises tungsten and is supported by the catalyst support material.

A twentieth aspect of the present disclosure may comprise the nineteenth aspect where the catalyst support material has a specific surface area of from 800 square meters per gram to 1000 square meters per gram.

A twenty-first aspect of the present disclosure may comprise either of the nineteenth or twentieth aspects where the catalyst support material has an average particle size of from 1 millimeter to 5 millimeters.

A twenty-second aspect of the present disclosure may comprise any of the nineteenth through twenty-first aspects where the catalyst support material has a pore volume of from 0.01 cubic centimeters per gram to 1 cubic centimeter per gram.

A twenty-third aspect of the present disclosure may comprise any of the nineteenth through twenty-second aspects where the catalyst support material has a pore density of from 0.01 grams per cubic centimeter to 1 gram per cubic centimeter.

A twenty-fourth aspect of the present disclosure may comprise any of the nineteenth through twenty-third aspects where the catalyst support material comprises activated carbon.

A twenty-fifth aspect of the present disclosure may comprise any of the nineteenth through twenty-fourth aspects where the catalytically active compound comprises tungsten oxide.

It should now be understood that various aspects of the present disclosure are described and such aspects may be utilized in conjunction with various other aspects.

It is noted that one or more of the following claims utilize the term “where” as a transitional phrase. For the purposes of defining the present disclosure, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

Having described the subject matter of the present disclosure in detail and by reference to specific embodiments, it is noted that the various details described in this disclosure should not be taken to imply that these details relate to elements that are essential components of the various embodiments described in this disclosure, even in cases where a particular element is illustrated in each of the drawings that accompany the present description. Rather, the appended claims should be taken as the sole representation of the breadth of the present disclosure and the corresponding scope of the various embodiments described in this disclosure. Further, it will be apparent that modifications and variations are possible without departing from the scope of the appended claims.

1. A metathesis catalyst comprising:
80 weight percent to 99 weight percent catalyst support material, based on the total weight of the metathesis catalyst, where the catalyst support material comprises carbon; and
1 weight percent to 20 weight percent catalytically active compound, based on the total weight of the metathesis catalyst, where the catalytically active compound comprises tungsten and is supported by the catalyst support material.

2. The metathesis catalyst of claim 1, where the catalyst support material has a specific surface area of from 800 square meters per gram to 1000 square meters per gram.

3. The metathesis catalyst of claim 1, where the catalyst support material has an average particle size of from 1 millimeter to 5 millimeters.

4. The metathesis catalyst of claim 1, where the catalyst support material has a pore volume of from 0.01 cubic centimeters per gram to 1 cubic centimeter per gram.

5. The metathesis catalyst of claim 1, where the catalyst support material comprises activated carbon.

6. The metathesis catalyst of claim 1, where the catalytically active compound comprises tungsten oxide.

7. A method of producing propene from a feedstock comprising butene, the method comprising:
joule heating a metathesis catalyst to an operational temperature; and
contacting the feedstock with the metathesis catalyst, where:
the metathesis catalyst comprises:
80 weight percent to 99 weight percent catalyst support material, based on the total weight of the metathesis catalyst, where the catalyst support material comprises carbon; and
1 weight percent to 20 weight percent catalytically active compound, based on the total weight of the metathesis catalyst, where the catalytically active compound comprises tungsten and is supported by the catalyst support material;
joule heating the metathesis catalyst comprises inserting one or more electrodes into a catalyst bed comprising the metathesis catalyst, and applying an electric current from the one or more electrodes to at least a catalyst support material of the metathesis catalyst such that from 20 watts to 40 watts are delivered to the catalyst support material; and
contacting of the feedstock with the metathesis catalyst at the operational temperature causes at least a portion of the butene in the feedstock to undergo a metathesis reaction to produce propene.

8-9. (canceled)

10. The method of claim 7, where the operational temperature is from 400 degrees Celsius to 600 degrees Celsius.

11. The method of claim 7, where the catalyst support material has a specific surface area of from 800 square meters per gram to 1000 square meters per gram.

12. The method of claim 7, where the catalyst support material has an average particle size of from 1 millimeter to 5 millimeters.

13. The method of claim 7, where the catalyst support material has a pore volume of from 0.01 cubic centimeters per gram to 1 cubic centimeter per gram.

14. A method of producing propene from a feedstock comprising butene, the method comprising:
joule heating a metathesis catalyst to an operational temperature; and
contacting the feedstock with the metathesis catalyst, where:
joule heating the metathesis catalyst comprises inserting one or more electrodes into a catalyst bed comprising the metathesis catalyst, and applying an electric current from the one or more electrodes to at least a catalyst support material of the metathesis catalyst such that from 20 watts to 40 watts are delivered to the catalyst support material; and
the contacting of the feedstock with the metathesis catalyst at the operational temperature causes at least a portion of the butene in the feedstock to undergo a metathesis reaction to produce propene.

15. (canceled)

16. The method of claim 14, where the metathesis catalyst comprises:
80 weight percent to 99 weight percent catalyst support material, based on the total weight of the metathesis catalyst, where the catalyst support material comprises carbon; and
1 weight percent to 20 weight percent catalytically active compound, based on the total weight of the metathesis catalyst, where the catalytically active compound comprises tungsten and is supported by the catalyst support material.

17. The method of claim 16, where the catalyst support material has a specific surface area of from 800 square meters per gram to 1000 square meters per gram.

18. The method of claim 16, where the catalyst support material has an average particle size of from 1 millimeter to 5 millimeters.

19. The method of claim 16, where the catalyst support material has a pore volume of from 0.01 cubic centimeters per gram to 1 cubic centimeter per gram.

20. (canceled)
